(19) United States
(12) Patent Application Publication
(10) Pub. No.: US 2026/0124172 A1
(43) Pub. Date: May 7, 2026

(54) Use of cembrane-type diterpene compounds in the preparation of medicament for preventing or treating inflammasome mediated inflammatory disorders

(71) Applicant: Chang Gung University of Science and Technology, Taoyuan City (TW)

(72) Inventors: Tsong-Long Hwang, Taoyuan City (TW); Jyh-Horng Sheu, Kaohsiung City (TW); Yu-Chia Chang, Taoyuan City (TW); I-Ying Liu, Taoyuan City (TW); Yu-Li Chen, Taoyuan City (TW)

(73) Assignee: Chang Gung University of Science and Technology, Taoyuan City (TW)

(21) Appl. No.: 19/372,322

(22) Filed: Oct. 29, 2025

Related U.S. Application Data
(60) Provisional application No. 63/714,862, filed on Nov. 1, 2024.

(51) Int. Cl. A61K 31/366; A61K 31/365; A61P 17/06; A61P 37/06

(52) U.S. Cl. CPC A61K 31/366; A61K 31/365; A61P 17/06; A61P 37/06

(57) ABSTRACT

A method for preventing or treating inflammasome-mediated inflammatory disorders, which includes administration of an effective amount of cembrane-type diterpene compound to a subject in need thereof or a subject suffering from inflammation mediated by inflammasome, the cembrane-type diterpene is isolated from soft coral Sinularia flexibilis or Sinularia manaarensis, and the cembrane-type diterpene is cembranolide.

### CROSS REFERENCE TO RELATED APPLICATION

This application claims the benefit of U.S. Provisional Patent Application No. 63/714,862 filed on Nov. 1, 2024, which is incorporated by reference herein in its entirety.

### FIELD OF THE INVENTION

The present invention relates to a method for preventing or treating inflammasome mediated inflammatory disorders in a subject, particularly, a method for preventing or treating inflammasome mediated inflammatory disorders in a subject by administering cembranolide compounds including dehydrosinulariolide, dihydroaustrasulfone alcohol, dihydrosinularin, 5-epi-Sinuleptolide, 11-epi-Sinulariolide acetate, sinularin, sinulariolide, or sinuleptolide.

### BACKGROUND OF THE INVENTION

Nucleotide-binding oligomerization domain-like receptor family pyrin domain containing 3 (NLRP3) inflammasome is a multiprotein complex composed of apoptosis-associated speck-like protein containing a CARD (ASC) and pro-caspase-1, when the cells are stimulated to start the activation of NLRP3 inflammasomes, NLRP3, ASC and pro-caspase-1 undergo oligomerization, and ASC will condense to form ASC specks, initiate pro-caspase-1 self-cleavage to produce activated caspase-1, which in turn promotes the maturation of inflammatory factors and triggers a special type of lytic programmed cell death. It is called pyroptosis.

The expression and function of NLRP3 inflammasomes are regulated by two steps: priming step (signal I) and activation step (signal II). Priming step (signal I) provides the substances needed by NLRP3 inflammasomes. During the priming step (signal I), macrophage membrane surface receptors recognize pathogen-associated molecular patterns (PAMPs) released from infected or damaged cells, activate the nuclear factor kappa-light-chain-enhancer of activated B cells (NF-κB), and promote the transcription and translation of NLRP3 inflammasome-associated proteins. Lipopolysaccharides (LPS) on the surface of bacteria are common PAMPs, which activate NF-κB through Toll-like receptor-4 (TLR4) and increase the expression of NLRP3, pro IL-1β and pro IL-18 proteins in cells.

In the activation step of NLRP3 inflammasomes, many substances are known to act as NLRP3 inflammasome activators to generate activated NLRP3 inflammasomes through different pathways in cells. Adenosine triphosphate (ATP), monosodium urate (MSU), imiquimod (IMQ) and nigericin are common NLRP3 inflammasome activators, which can cause intracellular potassium ion outflow, reactive oxygen species (reactive oxygen species; ROS) generation and lysosomal rupture, which activates NLRP3 inflammasomes and causes pro-caspase-1 to self-cleavage and release caspase-1.

Natural compounds are advantageous for their abundant supplies and diverse skeletons, and they are important bases for drug development. From 1981 till 2019, nearly half of the new FDA-approved drugs were derived from natural products or their derivatives, for example, cocaine-derived narcotics, morphine-derived analgesics, vincristine, doxorubicin and paclitaxel for treating cancers, and fungus-derived penicillin as antibiotics. Therefore, the present invention actively investigates which natural compounds have the effect of inhibiting NLRP3 inflammasomes and the potential to be further developed into novel drugs for the treatment or prevention of inflammasome-mediated inflammatory diseases.

### SUMMARY OF THE INVENTION

The present invention is related to a method for preventing or treating inflammasome-mediated inflammatory disorders, which comprises administration of a cembrane-type diterpene compound to a subject in need thereof or to a subject suffering from inflammation mediated by inflammasome.

In a preferred manner, the said cembrane-type diterpene is cembranolide.

In a most preferred manner, the said cembranolide comprises dehydrosinulariolide, dihydroaustrasulfone alcohol, dihydrosinularin, 5-epi-sinuleptolide, 11-epi-sinulariolide acetate, sinularin, sinulariolide or sinuleptolide.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the screening results of NLRP3 inflammasome inhibitors, wherein the NLRP3 inflammasome inhibitors are cembrane-type diterpene compounds. Symbol description: “+” refers to receive treatment, “−” refers to not receive treatment, ** refers to statistically significant compared with the nigericin activation alone group (p<0.01); *** refers to statistically significant compared to the nigericin activation alone group (p<0.001).

FIG. 2 shows the chemical formula of sinularin.

FIG. 3A and FIG. 3B show the cytotoxicity test results for sinularin. LPS means lipopolysaccharide. The results are presented as mean±standard error (n=6 to 8). Symbol description: “+” refers to receive treatment, “−” refers to not receive treatment.

FIG. 4 shows the experiment process of pretreated sinularin inhibiting IL-1β and IL-18 release and pyroptosis in LPS-primed d-THP-1 cells. LPS means lipopolysaccharide, IMQ means imiquimod, and MSU means monosodium urate.

FIG. 5A to FIG. 5C show that pretreatment of sinularin significantly and dose-dependently inhibits the release of IL-1ß from LPS-primed d-THP-1 treated with different activators. LPS means lipopolysaccharide, IMQ means imiquimod, and MSU means monosodium urate. The results are presented as mean±standard error (n=6 to 8). FIG. 5A shows the IL-1β release results of d-THP-1 treated with nigericin as an activator. FIG. 5B shows the IL-1ß release results of d-THP-1 treated with IMQ as an activator. FIG. 5C shows the IL-1β release results of d-THP-1 treated with MSU as an activator. Symbol description: “+” refers to receive treatment, “−” refers to not receive treatment, * refers to statistically significant compared with activator treatment alone group (p<0.05); ** refers to statistically significant compared to the activator treatment alone group (p<0.01); *** refers to statistically significant compared to the activator-treated alone group (p<0.001).

FIG. 6A to FIG. 6C show that pretreatment of sinularin significantly and dose-dependently inhibits the release of IL-18 from LPS-primed d-THP-1 treated with different activators. LPS means lipopolysaccharide, IMQ means imiquimod, and MSU means monosodium urate. FIG. 6A shows the IL-18 release results of d-THP-1 treated with nigericin as an activator; FIG. 6B shows the IL-18 release results of d-THP-1 treated with IMQ as an activator; FIG. 6C shows the IL-18 release results of d-THP-1 treated with MSU as an activator. The results are presented as mean±standard error (n=7 to 8). Symbol description: “+” refers to receive treatment, “−” refers to not receive treatment, * refers to statistically significant compared with activator treatment alone group (p<0.05); ** refers to statistically significant compared to the activator treatment alone group (p<0.01); *** refers to statistically significant compared to the activator-treated alone group (p<0.001).

FIG. 7A to FIG. 7C show the result of sinularin pretreatment inhibits the release of lactate dehydrogenase (LDH) of pyroptosis. LPS means lipopolysaccharide, IMQ means imiquimod, and MSU means monosodium urate. FIG. 7A shows the LDH release results of LPS-primed d-THP-1 using nigericin as an activator; FIG. 7B shows the LDH release results of LPS-primed d-THP-1 using IMQ as an activator; FIG. 7C shows the LDH release results of LPS-primed d-THP-1 using MSU as an activator. The results are presented as mean±standard error (n=6 to 7). Symbol description: “+” refers to receive treatment, “−” refers to not receive treatment, * refers to statistically significant compared with activator treatment alone group (p<0.05); ** refers to statistically significant compared to the activator treatment alone group (p<0.01); *** refers to statistically significant compared to the activator-treated alone group (p<0.001).

FIG. 8 shows experiment process of sinularin inhibits IL-1β and IL-18 release and pyroptosis in LPS-primed d-THP-1 cells. LPS means lipopolysaccharide, IMQ means imiquimod, and MSU means monosodium urate.

FIG. 9A to FIG. 9C show that sinularin significantly and dose-dependently inhibits IL-1β release caused by NLRP3 inflammasomes in LPS-primed d-THP-1 cells. LPS means lipopolysaccharide, IMQ means imiquimod, and MSU means monosodium urate. FIG. 9A shows the IL-1β release results of LPS-primed d-THP-1 using nigericin as an activator; FIG. 9B shows the IL-1β release results of LPS-primed d-THP-1 using IMQ as an activator; FIG. 9C shows the IL-1β release results of LPS-primed d-THP-1 using MSU as an activator. The results are presented as mean±standard error (n=6 to 7). Symbol description: “+” refers to receive treatment, “−” refers to not receive treatment, ** refers to statistically significant compared to the activator treatment alone group (p<0.01); *** refers to statistically significant compared to the activator-treated alone group (p<0.001).

FIG. 10A to FIG. 10C show that sinularin significantly and dose-dependently inhibits IL-18 release caused by NLRP3 inflammasomes in LPS-primed d-THP-1 cells. LPS means lipopolysaccharide, IMQ means imiquimod, and MSU means monosodium urate. FIG. 10A shows the IL-18 release results of LPS-primed d-THP-1 using nigericin as an activator; FIG. 10B shows the IL-18 release results of LPS-primed d-THP-1 using IMQ as an activator; FIG. 10C shows the IL-18 release results of LPS-primed d-THP-1 using MSU as an activator. The results are presented as mean±standard error (n=6 to 7). Symbol description: “+” refers to receive treatment, “−” refers to not receive treatment, * refers to statistically significant compared with activator treatment alone group (p<0.05); ** refers to statistically significant compared to the activator treatment alone group (p<0.01); *** refers to statistically significant compared to the activator-treated alone group (p<0.001).

FIG. 11A to FIG. 11C show that sinularin inhibits the LDH production of pyroptosis by NLRP3 inflammasomes in LPS-primed d-THP-1 cells. LPS means lipopolysaccharide, IMQ means imiquimod, and MSU means monosodium urate. FIG. 11A shows the LDH release results of LPS-primed d-THP using nigericin as an activator; FIG. 11B shows the LDH release results of LPS-primed d-THP using IMQ as an activator. FIG. 11C shows the LDH release results of LPS-primed d-THP using MSU as an activator. The results are presented as mean±standard error (n=6 to 7). Symbol description: “+” refers to receive treatment, “−” refers to not receive treatment, ** refers to statistically significant compared to the activator treatment alone group (p<0.01); *** refers to statistically significant compared to the activator-treated alone group (p<0.001).

FIG. 12A to FIG. 12B show the results of sinularin inhibiting cell morphological changes caused by pyroptosis. IMQ means imiquimod, and MSU means monosodium urate. FIG. 12A shows the cell morphological changes under the treatment of nigericin as an activator; FIG. 12B shows cell morphological changes under the treatment of IMQ as an activator. Symbol description: “+” refers to receive treatment, “−” refers to not receive treatment.

FIG. 13 shows the results of sinularin inhibiting ASC speck formation. LPS means lipopolysaccharide, IMQ means imiquimod. The results are presented as mean±standard error (n=6). Symbol description: “+” refers to receive treatment, “−” refers to not receive treatment, *** refers to statistically significant compared to the activator-treated alone group (p<0.001).

FIG. 14 shows the results of sinularin inhibiting the generation of intracellular reactive oxygen species (ROS). LPS means lipopolysaccharide, IMQ means imiquimod. The results are presented as mean±standard error (n=6). Symbol description: “+” refers to receive treatment, “−” refers to not receive treatment, *** refers to statistically significant compared to the activator-treated alone group (p<0.001).

FIG. 15 shows the western blotting results of sinularin inhibiting the expression of NLRP3 inflammasome-related proteins. LPS means lipopolysaccharide. The results are presented as mean±standard error (n=6). Symbol description: “+” refers to receive treatment, “−” refers to not receive treatment, * refers to statistically significant compared with activator treatment alone group (p<0.05); ** refers to statistically significant compared to the activator treatment alone group (p<0.01); *** refers to statistically significant compared to the activator-treated alone group (p<0.001).

FIG. 16A and FIG. 16B show the result of sinularin mitigating acute liver injury (ALI) in mice. LPS means lipopolysaccharide, D-GalN means D-galactosamine, LPS/D-GalN refers to induction of ALI without any treatment, H&E means hematoxylin-eosin staining, F4/80 is a biomarker of macrophages, and NLRP3 means nucleotide-binding oligomerization domain-like receptor family pyrin domain containing 3. FIG. 16A shows the appearance of the liver of ALI mice; FIG. 16B shows the results of liver tissue section from ALI mice. The results are presented as mean±standard error (n=6). Symbol description: “+” refers to receive treatment, “−” refers to not receive treatment.

FIG. 17 shows the effects of sinularin on serum biochemical indicators in ALI mice. Vehicle refers to the administration of solvents, LPS means lipopolysaccharide, D-GalN means D-galactosamine, LPS/D-GalN refers to induction of ALI without any treatment, GOT means glutamic oxaloacetic transaminase, GPT means glutamic pyruvic transaminase, BUN means blood urea nitrogen, and CRE means creatinine. The results are presented as mean±standard error (n=6).

FIG. 18A to FIG. 18C show the result of skin application of sinularin mitigates psoriasis in mice. IMQ means imiquimod. FIG. 18A shows the skin appearance results of psoriasis mice (n=3 to 4); FIG. 18B shows the skin tissue section results of psoriasis mice (n=3 to 4); FIG. 18C shows trans epidermal water loss (TEWL) results (n=3 to 4). Sham indicates sham group, and the IMQ indicates imiquimod for inducing psoriasis without any treatment.

### DETAILED DESCRIPTION OF THE INVENTION

In the present invention, cembrane-type diterpene compounds are isolated from soft corals and their inhibitory effect on NLRP3 inflammasomes are tested.

The cembrane-type diterpene compounds in the present invention are preferably isolated from soft coral Sinularia flexibilis or soft coral Sinularia manaarensis.

The present invention is related to a use of cembrane-type diterpene compounds in the preparation of medicament for preventing or treating inflammasome mediated inflammatory disorders or a method for preventing or treating inflammasome mediated inflammatory disorders in a subject suffering from inflammatory disorders mediated by inflammasome.

The present invention is related to a use of drug for preventing or treating inflammasome mediated inflammatory disorders, which comprises administration of an effective amount of cembrane-type diterpene compound to a subject in need thereof.

The cembrane-type diterpene compounds in the present invention are preferably cembranolide. Most preferred, the cembrane-type diterpene compounds in the present invention include dehydrosinulariolide, dihydroaustrasulfone alcohol, dihydrosinularin, 5-epi-sinuleptolide, 11-epi-sinulariolide acetate, sinularin, sinulariolide, or sinuleptolide.

The NLRP3 inflammasome mediated inflammatory disorders comprise, but not limited to, stroke, type 2 diabetes (T2DM), atherosclerosis, severe acute respiratory syndrome coronavirus 2 (SARS-COV-2), acute respiratory distress syndrome (ARDS), acute lung injury (ALI), inflammatory bowel disease, atopic dermatitis, psoriasis, alopecia areata, systemic lupus erythematosus (SLE), lupus nephritis (LN) caused by SLE, gouty arthritis (GA), rheumatic disease, human autoimmune skin disease, Alzheimer's disease, Parkinson's disease, Huntington's disease, or cryopyrin-associated periodic syndrome (CAPS).

The CAPS includes familial cold autoinflammatory syndrome (FCAS), Muckle-Well syndrome (MWS), or chronic infantile neurological cutaneous articular syndrome (CINCA).

In the present invention, the subject of the present invention is human (e.g. infant, toddler, teenager, young adult, middle-aged adult or elderly), rodent (e.g. mice, rat) or mammal (e.g. companion animal such as dog, cat, rabbit, marten; economic animals such as horse, sheep, cow, pig). In a preferred manner, the subject of the present invention is human or non-human mammal. The subject of the present invention is most preferred human.

In the present invention, the effective amount of the cembrane-type diterpene compounds is 0.001 mg/kg-body weight to 100 mg/kg-body weight.

To take a step further, the effective amount of the cembrane-type diterpene compounds of the present invention can be 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 21, 22, 25, 26, 27, 28, 29, 30, 31, 32, 33, 34, 35, 36, 37, 38, 39, 40, 41, 42, 43, 44, 45, 46, 47, 48, 50, 51, 52, 53, 54, 55, 56, 57, 58, 59, 60, 61, 62, 63, 64, 65, 66, 67, 68, 69, 70, 71, 72, 73, 75, 76, 77, 78, 79, 80, 81, 82, 83, 84, 85, 86, 87, 88, 89, 90, 91, 92, 93, 94, 95, 96, 97, 98, 100 mg/kg-body weight.

The effective amount of the cembrane-type diterpene compounds of the present invention in mice is 1 mg/kg·bw to 100 mg/kg·bw. Effective amount of the cembrane-type diterpene compounds of the present invention in mice is preferably 5 mg/kg·bw to 75 mg/kg·bw. Most preferred, the effective amount of the cembrane-type diterpene compounds of the present invention is 5 mg/kg·bw to 10 mg/kg·bw for injection and 25 mg/kg·bw to 50 mg/kg·bw for topical application.

The effective amount of the present invention can be conversed to equivalent effective amount of different species subject according to the formula and the safety factor provided in Guidance for Industry: Establishing the Maximum Safe Starting Doses in Initial Clinical Trials for Therapeutics in Adult Healthy Volunteers, U.S. Department of Health and Human Services, Food and Drug Administration Center for Drug Evaluation and Research (CDER), July 2005 published by FDA.

In the present invention, the equivalent dose of the cembrane-type diterpene compounds administering to a 60 kg body weight human is 0.08 mg/kg·bw to 8.1 mg/kg·bw. The equivalent dose of the cembrane-type diterpene compounds administering to a 60 kg body weight human is preferably 0.4 mg/kg·bw to 6.1 mg/kg·bw. Most preferred, the equivalent dose of the cembrane-type diterpene compounds administering to a 60 kg body weight human is 0.4 mg/kg·bw to 0.8 mg/kg·bw for injection and 2 mg/kg·bw to 4.1 mg/kg·bw for topical application. If the safety factor is further considered, the recommended starting dose is one-tenth of the human equivalent dose.

The present can also comprise a pharmaceutically acceptable carrier, particularly can further comprise predetermined solvents or oils, PH adjuster and if desired, can further comprise a dispersant. Examples of solvents used in the present invention include, but are not limited to, water, ethanol, isopropanol, 1,3-butanediol, propylene glycol, glycerin, etc. Examples of oils used in the present invention are selected from the group consisting of, but are not limited to, corn oil, sesame oil, flaxseed oil, cottonseed oil, soybean oil, peanut oil, mono-glycerides, di-glycerides, tri-glycerides, mineral oil, squalene, jojoba oil, olive oil, evening primrose oil, borage oil, grape seed oil, coconut oil, sunflower oil, shea butter, and any combinations thereof. Solvents and oils can be used alone or in any combinations thereof.

Examples of useful dispersants that are beneficial to the present invention can include, but are not limited to, lecithin, organic monoglycerides, sorbitan fatty acid esters, polyoxyethylene fatty acid esters, sorbitan stearate, etc. These raw materials can also be used alone or in any combinations thereof.

In a preferred manner, the drug of the present invention is prepared as external preparations, examples of the external preparations can include, but are not limited to, creams, ointments, gels, wash lotions or patches, etc., or inhalants, aerosols, suppositories, etc. The drug of the present invention is also administered by injection in the present invention.

When the drug is used as an external preparation, an appropriate external skin preparation can be used as a base material, and an aqueous solution, a non-aqueous solvent, a suspension, an emulsion or a lyophilized preparation, etc., can be used and sterilized according to known methods. The compositions in the form of gels, creams and ointments can be prepared according to the form of the composition by using known methods, and by addition of known softeners, emulsifiers and thickeners or other materials known in the art. The gel-form composition can be prepared, for example, by addition of a softener such as trimethylolpropane, polyethylene glycol and glycerol, for example, a solvent of propylene glycol, ethanol and isocetyl alcohol, and pure water.

### EMBODIMENTS

The examples below are non-limiting and are merely representative of various aspects and features of the present invention.

### Compounds to be Tested

The cembrane-type diterpene compounds of the present invention were provided by Jyh-Horng Sheu, professor of department of marine biotechnology and resources, National Sun Yat-sen University. They were isolated from soft coral Sinularia flexibilis or soft coral Sinularia manaarensis. The cembrane-type diterpene compounds include dehydrosinulariolide, dihydroaustrasulfone alcohol, dihydrosinularin, 5-epi-Sinuleptolide, 11-epi-Sinulariolide acetate, sinularin, sinulariolide, or sinuleptolide.

### Pharmaceutical and Material Reagents

The pharmaceutical and materials of the present invention are shown in Table 1.

TABLE 1

| Product name | Brand | Origin |
|---|---|---|
| 2-Mercaptoethanol (β-ME) | Gibco | U.S.A. |
| Apoptosis-associated speck-like | Santa Cruz | U.S.A. |
| protein containing a CARD (ASC) | | |
| antibody (B-3) Alexa Fluo 488 | | |
| NLRP3 polyclonal antibody (pAb) | Abcam | UK |
| F4/80 monoclonal antibody (mAb) | Abcam | UK |
| Interleukin 1β (IL-1β) pAb | Abcam | UK |
| Caspase-1 pAb | Abcam | UK |
| Antibiotic-antimycotic solution | Gibco | U.S.A. |
| (penicillin, streptomycin, and | | |
| amphotericin B) | | |
| Bovine serum albumin (BSA) | Sigma-Aldrich | U.S.A. |
| CytoTox 96 ® non-radioactive | Promega | U.S.A. |
| cytotoxicity assay kits | | |
| Cleaved-Caspase-1 antibody | Cell Signaling | U.S.A. |
| Cleaved- IL-1β antibody | Cell Signaling | U.S.A. |
| Clarity ™ Western enhanced | Bio-Rad | U.S.A. |
| chemiluminescence (ECL) substrate | | |
| Dimethyl sulfoxide (DMSO) | Sigma-Aldrich | U.S.A. |
| D-galactosamine (D-GalN) | Sigma-Aldrich | U.S.A. |
| ELISA MAX ™ Deluxe Set Mouse | BioLegend | U.S.A. |
| TNF-α | | |
| Fetal bovine serum (FBS) | Gibco | U.S.A. |
| FL-GSDMD antibody | Cell Signaling | U.S.A. |
| GOT slide | Fujifilm | Japan |
| GPT slide | Fujifilm | Japan |
| BUN slide | Fujifilm | Japan |
| CRE slide | Fujifilm | Japan |
| GAPDH antibody | Cell Signaling | U.S.A. |
| Glutaraldehyde | Sigma-Aldrich | U.S.A. |
| Goat anti-Rabbit IgG (H + L) highly | Thermo Fisher | U.S.A. |
| cross-adsorbed secondary antibody | | |
| Hoechst 33342 | ImmunoChemistry | U.S.A. |
| Human IL-18 ELISA kit | R&D system | U.S.A. |
| Human IL-1β ELISA kit | R&D system | U.S.A. |
| Imiquimod (R837) | InvivoGen | U.S.A. |
| Imiquimod, Aldara ™ 5% cream | MEDA | UK |
| Difco ™ LB Broth, Miller (Luria | BD biosciences | U.S.A. |
| Bertani) | | |
| Difco ™ LB AGAR, Miller (Luria | BD biosciences | U.S.A. |
| Bertani) | | |
| Monosodium urate crystals (MSU) | InvivoGen | U.S.A. |
| Nigericin sodium salt | Sigma-Aldrich | U.S.A. |
| NT-GSDMD antibody | Cell Signaling | U.S.A. |
| Osmium tetroxide (OsO4) | Sigma-Aldrich | U.S.A. |
| Paraformaldehyde (20% PFA) | Electron microscopy | U.S.A. |
| | sciences | |
| Pierce ™ BCA protein assay kit | Thermo | U.S.A. |
| Pro-IL-1β antibody | Cell Signaling | U.S.A. |
| Pro-Caspase-1 antibody | Cell Signaling | U.S.A. |
| Phorbol 12-myristate 13-acetate | Sigma-Aldrich | U.S.A. |
| (PMA) | | |
| Phosphate-buffered saline (PBS) | Sigma-Aldrich | U.S.A. |
| PhosSTOP EASYpack | Roche | Switzerland |
| Poly-Lysine coated coverslip | Corning | U.S.A. |
| RPMI-1640 | ATCC | U.S.A. |
| RPMI-1640 (without phenol red) | Gibco | U.S.A. |
| THP-1 cell line (TIB202 ™) | ATCC | U.S.A. |
| Triton X-100 | Sigma-Aldrich | U.S.A. |
| Tween-20 | Sigma-Aldrich | U.S.A. |
| Tannic acid | Sigma-Aldrich | U.S.A. |
| Ultra pure lipopolysaccharide | InvivoGen | U.S.A. |
| (UP-LPS, E. coli O111:B4) | | |

### Cells Subculture

THP-1 cells (hereinafter THP-1) are a commonly used human monocytic cell line that is isolated from the peripheral blood of patients with acute monocytic leukemia and were purchased from the American Type Culture Collection (ATCC). THP-1 was cultured in RPMI-1640 medium containing 10% fetal bovine serum (FBS), 1% antibiotic-antimycotic solution, 0.1% β-mercaptoethanol (β-ME), and phenol red indicator. Cells were subcultured in a T75 flask, then were cultured at a cell concentration of 3*10⁵ cells per milliliter in a cell culture incubator (37° C., 5% CO2). When the cell grows to a density of 8*10⁵-1*10⁶ cells/mL, centrifugation (200×g, 25° C., 5 minutes) was required to replace the culture medium for subculture.

### Cellular Differentiation

Culture medium was replaced with serum-free (FBS-free) RPMI-1640 medium containing 1% antibiotic-antimycotic solution when THP-1 grew to a cell concentration of approximately 1*10⁶ cells per milliliter. Serum-free RPMI-1640 medium containing 100 nM phorbol 12-myristate 13-acetate (PMA) was added into a 24-well dish, then equal volume of cell suspension was slowly added and mixed thoroughly, THP-1 was incubated for 24 hours in a cell culture incubator (37° C., 5% CO₂) to differentiate THP-1 into M1 macrophages (d-THP-1).

### Quantitative Analysis of Interleukin 1β (IL-1β) and Interleukin 18 (IL-18)

Human IL-1β and Human IL-18 were quantified using an Enzyme-linked immunosorbent assay kit (ELISA kit, purchased from the R&D system). A capture antibody diluted to 4 μg/mL in phosphate-buffered saline (PBS) was added into a 96-well dish and placed overnight at room temperature, and PBS containing 1% bovine serum albumin (BSA) will be added as a reagent diluent buffer for an hour at room temperature the next day. The cell supernatant was diluted with 1% BSA, the standard is serial diluted with 1% BSA, after 2 hours of reaction at room temperature, washed three times, and the remained wash buffer in the 96-well dish was finally dried with a paper towel, detection antibody was added and reacted at room temperature for 2 hours, washed three times, and Horseradish Peroxidase (HRP) conjugated streptavidin (streptavidin-HRP) was added, the reaction was protected from light for 20 minutes, washed three times. A substrate solution was added into each well, the reaction was protected from light for 20 minutes, and phosphoric acid (H₃PO₄) with a concentration of 1 M was finally added as a stop solution to stop the reaction. The reading value of wavelengths of 450 nm and 540 nm were measured with an ELISA reader, drew out the standard curve, found the formula, and other experimental reading values were then taken into the formula to calculate the values of IL-1β or IL-18.

Glibenclamide with a concentration of 20 M was used as a control group in the present study.

### Detection of Cytotoxicity

Lactic dehydrogenase (LDH) is a stable cytoplasmic enzyme that is released when cells are toxically stimulated or damaged by death. This principle can be used to detect indicators of the toxic effects of compounds in the supernatant on cells.

Cembrane-type diterpene compound, for example sinularin, was added to d-THP-1 cell for reaction for 4 hours or 8 hours, the supernatant was corrected to 96-well plate. LDH substrate was added, the reaction was then protected from light for 20 minutes and the absorbance was measured at wavelengths of 490 nm. The group that cells with Triton X-100 (0.1%) added for 30 minutes represented the total LDH released, and the percentage of cell survival was calculated.

Glibenclamide with a concentration of 20 μM was used as a control group in the present study.

### Inhibition Assay of Nucleotide-Binding Oligomerization Domain-Like Receptor Family Pyrin Domain Containing 3 (NLRP3) Inflammasome Activation-Induced Cell Pyroptosis

The mechanism of pyroptosis usually involves the activation of inflammatory caspase, and the activation of Nod-like receptor protein 3 (NLRP3) inflammasome causes the lysed GSDMD to form an active N-terminal GSDMD, thus forming holes in the cell membrane, which in turn causes swelling and rupture, leading to pyroptosis and the release of LDH.

The activation of NLRP3 inflammasome are defined by priming step (hereinafter referred to as signal I) and activating step (hereinafter referred to as signal II). In the present invention, lipopolysaccharide (LPS) was used as an activator of signal I, and nigericin, IMQ, or MSU was used as an activator of signal II.

In the present invention, cembrane-type diterpene compound to be tested (sinularin was taken as example) was added to d-THP-1 cells before or after LPS priming, different stimulator such as nigericin, imiquimod (IMQ) or monosodium urate crystals (MSU) was added after the reaction, then cells were cultured in a cell incubator (37° C., 5% CO₂) to test the inhibitory effect of the cembrane-type diterpene compound to inflammasome activation.

In the inflammasome inhibition experiment of the present invention, the d-THP-1 cells were divided into two groups, group I and group II, according to the administration time of cembrane-type diterpene compound. Group 1: d-THP-1 cells were treated with cembrane-type diterpene compounds before priming of LPS, and group 2: d-THP-1 cells were treated with cembrane-type diterpene compounds after priming of LPS.

The influence of signal I signal transduction could be eliminated when cells were treated with cembrane-type diterpene compounds after priming of LPS.

Glibenclamide with a concentration of 20 μM was used as a control group in the present study.

Experimental steps are described as following:

### Group 1:

After THP-1 cells differentiated into d-THP-1 cells, the supernatant was removed and replaced with serum-free RPMI-1640 medium containing different concentrations of cembrane-type diterpene compounds. After 30 minutes reaction in a cell culture incubator (37° C., 5% CO₂), 100 ng/ml of ultrapure LPS (UP-LPS) was added for 3 hours, and then activator such as nigericin, IMQ or MSU was added. After 1 hour (nigericin), 2 hours (IMQ) or 4 hours (MSU) reaction in a cell culture incubator (37° C., 5% CO₂), the supernatant was collected for subsequent experiments.

### Group 2:

After THP-1 cells differentiated into d-THP-1 cells, the supernatant was removed and replaced with serum-free RPMI-1640 medium containing 100 ng/ml of ultrapure UP-LPS for cell culture. After 3 hours reaction in a cell culture incubator (37° C., 5% CO₂), the supernatant was removed and replaced with serum-free RPMI-1640 medium without 100 ng/ml of ultrapure UP-LPS. After 30 minutes culturing in a cell culture incubator (37° C., 5% CO₂), different concentrations of cembrane-type diterpene compounds were added for 30 minutes, and then activator such as nigericin, IMQ or MSU was added, respectively. After 1 hour (nigericin), 2 hours (IMQ) or 4 hours (MSU) reaction in a cell culture incubator (37° C., 5% CO₂), the supernatant was collected for subsequent experiments.

### Fluorescence Detection of ASC Speck

d-THP-1 cells were treated with LPS for 3 hours, then the cells were reacted with cembrane-type diterpene compound (sinularin was taken as example) for 30 minutes, and then an activator (nigericin, IMQ or MSU) was added. After the reaction was completed, 4% paraformaldehyde (PFA) was added to the cells to fix the cells for 10 minutes. 0.1% Triton X-100 was taken for a further reaction at room temperature for 10 hours, washed three times with PBS (pH 7.4), replaced with PBS containing 5% BSA (pH 7.4) for an hour, washed with PBS three times, add anti-ASC primary antibody (1:500 dilution) was used for reaction at 4° C. overnight, washed with PBS three times, and replaced with goat anti-rabbit IgG Alexa Flour 488 secondary antibody (1:500 dilution), washed three times with PBS, 100 mg/mL Hoechst dye for 10 minutes, photographed with fluorescence microscope.

### Observation of Changes in Pyroptosis Morphology

d-THP-1 cells were treated with LPS for 3 hours, then the cells were reacted with cembrane-type diterpene compound (sinularin was taken as example) for 30 minutes, and then an activator (nigericin, IMQ, or MSU) was added. After the reaction was completed, 4% paraformaldehyde (PFA) was added to the cells to fix the cells for 10 minutes. 2.5% glutaraldehyde was used to fix cells at room temperature for 10 minutes, washed with PBS (pH 7.4) three times, and reacted overnight at 4° C. After 30 minutes reaction with 1% osmium tetroxide (OsO₄), double distilled water (ddH₂O) was used for washing, then 1% tannic acid was used for 30 minutes reductive effect. Dehydration was performed according to the serial ethanol concentration (30%, 50%, 70%, 80%, 90% and 100%), 100% ethanol was then used for dehydration three times, followed by cyclic drying in a superfluid critical dryer using carbon dioxide (CO₂) and ethanol. The samples were then fixed on an aluminum platform using graphene tape, and gold plating was performed using a gold atom ion sputterer under conditions of 1-2 mA for 90 seconds, and the cells were observed with a field-effect emission electron microscope.

### Fluorescence Detection of Mitochondrial Reactive Oxygen Species

d-THP-1 cells were treated with LPS for 3 hours, then the cells were reacted with cembrane-type diterpene compound (sinularin was taken as example) for 30 minutes, and then an activator (nigericin, IMQ, or MSU) was added. After the reaction was completed, the cell culture medium was renewed, 5 μM MitoSOX reagent and 200 μg/mL Hoechst dye were then added for 10 minutes, renewed the cell culture medium, and fluorescence microscopy was used to observe reactive oxygen species (ROS).

### Western Blotting

Serum-free medium containing 100 nM PMA was added to the 6-well culture plate, and then the cells were slowly added to the 6-well culture plate at a concentration of 2.5*10⁶ cells/mL, mixed with serum-free medium in equal volume and cultured in an incubator for 24 hours for cells differentiation. The supernatant was removed 24 hours post cell differentiation, replaced the medium to serum-free medium containing 100 ng/mL UP-LPS in the same volume for culture, and put into the incubator for 3 hours, replaced the medium to serum-free medium without UP-LPS for 30 minutes, and different concentrations of cembrane-type diterpene compound (sinularin was taken as example) for 30 minutes. Nigericin activator was then added for 30 minutes, removed the supernatant, washed the cells with PBS, placed the 6-well culture plate on ice and added an appropriate amount of radioimmunoprecipitation assay (RIPA) buffer, cells were scraped in the same direction with a spatula, stood for 30 minutes, and centrifuged it at 14,000×g at 4° C. for 30 minutes to collect the supernatant.

Proteins were separated according to the different sizes of proteins with primary structure by 12% or 15% polyacrylamide gel, the proteins were transferred to the nitrocellulose membrane, and then the protein transferred nitrocellulose membrane was shaken at room temperature with 5% BSA for one hour to block non-specific binding. Then the protein transferred nitrocellulose membrane was soaked in 5% BSA containing the primary antibody that can recognize the target protein, overnight reaction at 4° C., so that the primary antibody could bind to the target protein, and then Tris Buffered Saline with Tween 20 mixed buffer (TBST) was used to rinse the incompletely reacted antibody, and the protein transferred nitrocellulose membrane that has bound to the primary antibody was then soaked in 5% BSA containing the secondary antibody for an hour at room temperature. The secondary antibody used in the present invention was goat anti-Rabbit IgG (H+L) highly cross-adsorbed secondary antibody. Finally, the enhanced chemiluminescence (ECL) substrate used for western blot was added to rinse several times to generate luminescence, and the luminescence and fluorescence image analysis system (BIO-RAD ChemiDOC MP Imaging System) was used for quantitative analysis.

### Acute Liver Injury Mouse Animal Model

C57BL/6 mice with 7 to 10-week-old, 20-25 grams weight (purchased from BioLASCO Taiwan Co. Ltd) were used in the experiment. Mice were randomly divided into five groups, namely vehicle group (treated with solvent only), acute liver injury (ALI) control group (treated with 40 mg/kg-body weight LPS mixed with 500 mg/kg-body weight D-galactosamine (D-GalN)), drug control group (treated with 10 mg/kg-body weight sinularin), low-dosage drug treated group (treated with LPS/D-GalN+5 mg/kg-body weight sinularin), and high-dosage drug control group (treated with LPS/D-GalN+10 mg/kg-body weight sinularin).

After the mice were restrained, the tail of the mice was locally disinfected with alcohol, and then insulin needles were injected into the tail vein with 50 μL of solvent (a mixture of 10% dimethyl sulfoxide (DMSO), 10% tween-20 and 80% saline), 5 mg/kg-body weight sinularin or 10 mg/kg-body weight sinularin according to the groups. One hour later, mice were intraperitoneally injected 40 μg/kg-body weight of LPS and 500 mg/kg-body weight of D-GalN mixture to induce ALI or intraperitoneally injected 280 μL of saline. The experimental animals were euthanized after 5 hours, and blood and tissues were taken for analysis.

### Psoriasis Mouse Animal Model

C57BL/6 mice with 7 to 10-week-old, 20-25 grams weight (purchased from BioLASCO Taiwan Co. Ltd) were used in the experiment.

The hair on the back of the mouse was removed clean, after 2 days, 5% w/w IMQ cream (62.5 mg) was skin topically applied for 5 consecutive days to induce psoriasis. Animal weight was measured daily. On the second day to the fifth day, after skin topical administration of IMQ cream for 6 hours, mice were treated with sinularin or solvent by skin topical application. The skin topically applied solvent was 70% ethanol. On the sixth day, the experimental animals were anesthetized, and a digital camera and a handheld microscope (Mini ScopeV) were used to take close-up views of the skin condition on the back of the mice and to measure the loss of trans-epidermal water loss (TEWL) and the redness and swelling of the back.

### Testing of Serum Biochemical Value

Blood was collected from the heart and left at room temperature for 30 minutes, then serum was separated by centrifugation at 1,500×g at 4° C. for 15 minutes. Blood biochemical values, including glutamic oxaloacetic transaminase (GOT), glutamic pyruvic transaminase (GPT), blood urea nitrogen (BUN), and creatinine (CRE), were evaluated by using a fully automated dry biochemical analyzer (Dri-Chem NX500i, Fujifilm).

### Tissue Staining

Histology and immunohistochemical staining analysis were performed using the Benchmark XT automated section staining system (Ventana Medical Systems) and the OptiView 3,3′-diaminobenzidine immunohistochemical staining kit (OptiView DAB IHC Detection Kit) in accordance with the original operating manual.

The left lobe of the mouse liver was taken as the site for tissue section staining, and about 4 square centimeters (1.6 cm*2.5 cm) of the affected area was taken from the mouse skin for tissue sectioning and was completed on a 4 μm paraffin-embedded section. Routine staining of tissue sections was performed according to standard procedures. The immunohistochemistry (IHC) staining was performed with hematoxylin-eosin (H&E) staining and the use of F4/80 monoclonal antibody (F4/80 mAb), NLRP3 polyclonal antibody (pAb), rabbit Caspase-1 pAb, and interleukin-1β (IL-1β) pAb, and histomorphology was observed by optical microscope.

### Statistical Methods

All the experimental results were expressed as mean±S.E.M., and the statistical difference was compared using Student's t-test. When the p value is less than 0.05 (p<0.05), it is considered to be a statistically significant difference.

### Experimental Results

### Screen of NLRP3 Inflammasome Inhibitor

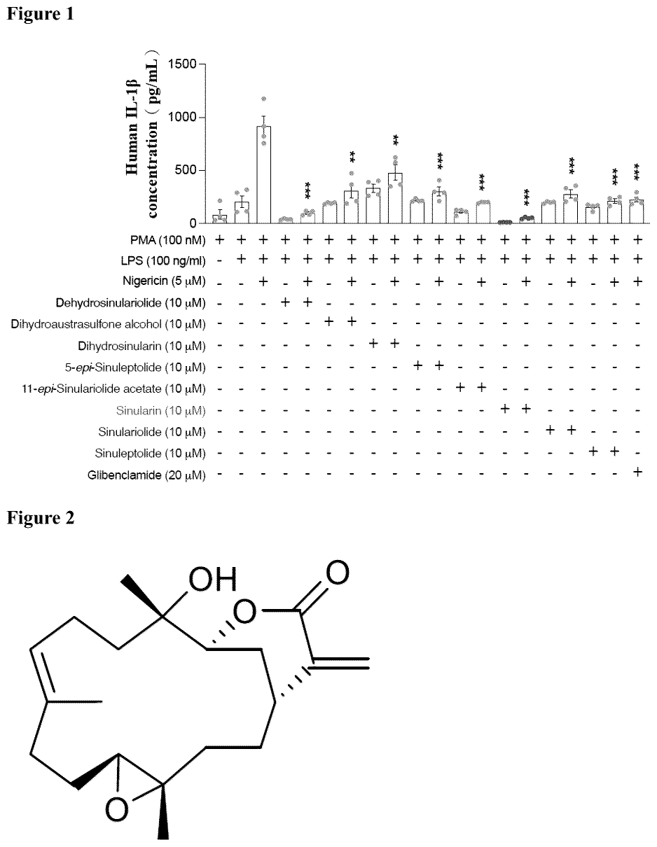

After LPS-priming of the d-THP-1 cells of the present invention, cembrane-type diterpene compound was added for screen of the NLRP3 inhibitors. The results are shown in FIG. 1, where sinularin shows the best NLRP3 inhibiting effect.

The chemical structure of sinularin is shown in FIG. 2.

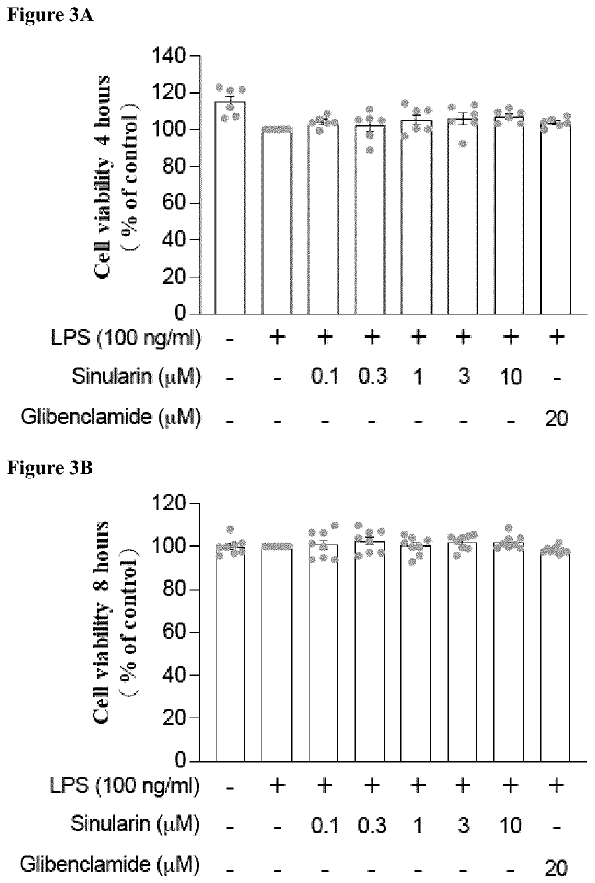

When cells are toxically stimulated or damaged by death, they lose the integrity of the cell membrane and release LDH. In the present invention, see FIG. 3A and FIG. 3B, LDH was not released from d-THP-1 cells treated with sinularin (0.1 to 10 μM) for 4 or 8 hours, sinularin is therefore presumed to be non-cytotoxic.

### Inhibition of IL-1β or IL-18 Release by Sinularin Pretreatment

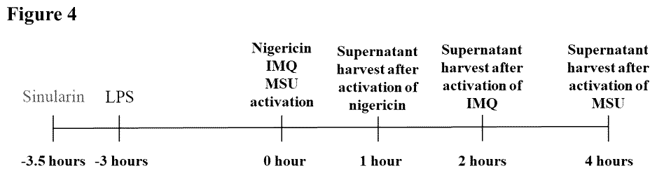

Different concentrations of sinularin (0.1 μM to 10 μM) were added to d-THP-1 cells before LPS priming, and different activators (nigericin, IMQ, or MSU) were followingly added, the experimental process is shown in FIG. 4.

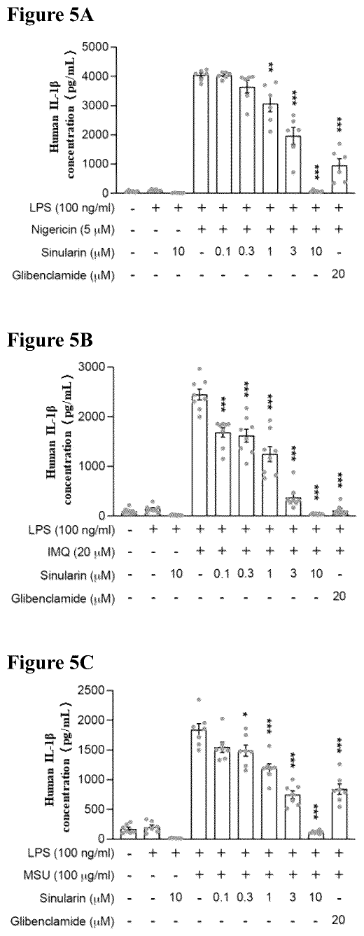

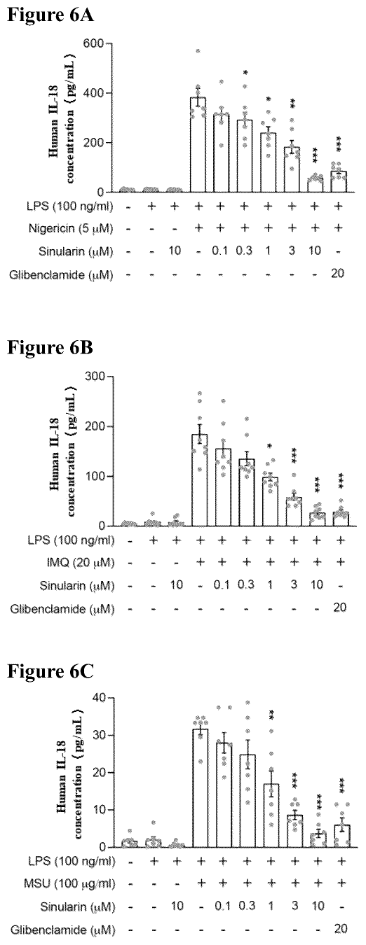

The IC₅₀ for inhibiting IL-1β release caused by nigericin, IMQ, and MSU were 1.05±0.25 μM (FIG. 5A), 2.61±1.39 μM (FIG. 5B), and 2.91±0.53 μM (FIG. 5C), respectively. The IC₅₀ for inhibiting IL-18 release caused by nigericin, IMQ, and MSU were 2.17±0.26 μM (FIG. 6A), 1.23±0.33 μM (FIG. 6B), and 1.03±0.37 μM (FIG. 6C), respectively.

### Inhibition of LDH Release of Pyroptosis by Sinularin Pretreatment

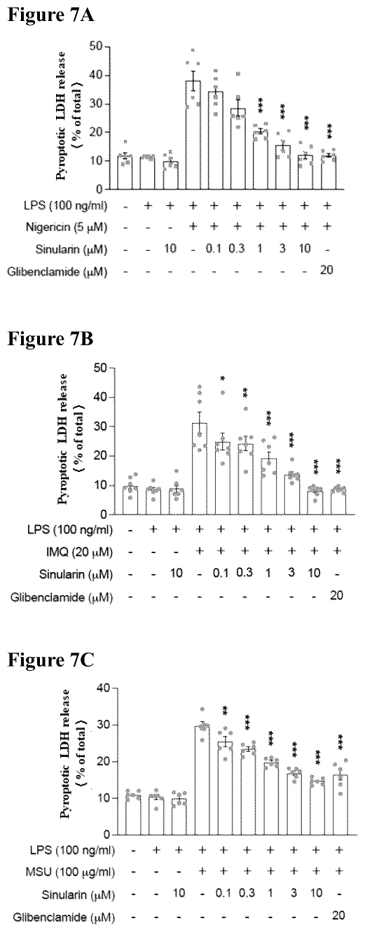

Different concentrations of sinularin (0.1 μM to 10 μM) were added to d-THP-1 cells before LPS priming, the results show that sinularin significantly and dose-dependently inhibited LDH release caused by different activators (nigericin, IMQ, or MSU). The IC₅₀ are 1.19±0.36 μM (FIG. 7A), 2.61±1.39 μM (FIG. 7B), and 2.91±0.53 μM (FIG. 7C), respectively.

Glibenclamide with a concentration of 20 μM was used as a control group in the present study.

### Inhibition of IL-1β or IL-18 Release Caused by NLRP3 Inflammasome by Sinularin

Sinularin was added to d-THP-1 cells after LPS priming to exclude its effect on signal I.

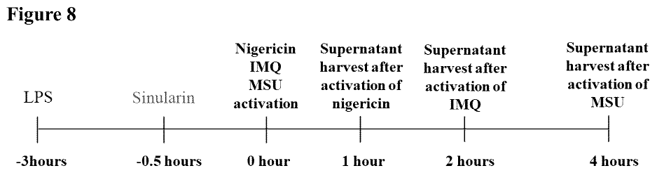

Sinularin (0.1 to 10 μM) was added to d-THP-1 cells after LPS priming, and the release of IL-1β and IL-18 were detected after the reaction of signal II activator. The experimental process is shown in FIG. 8.

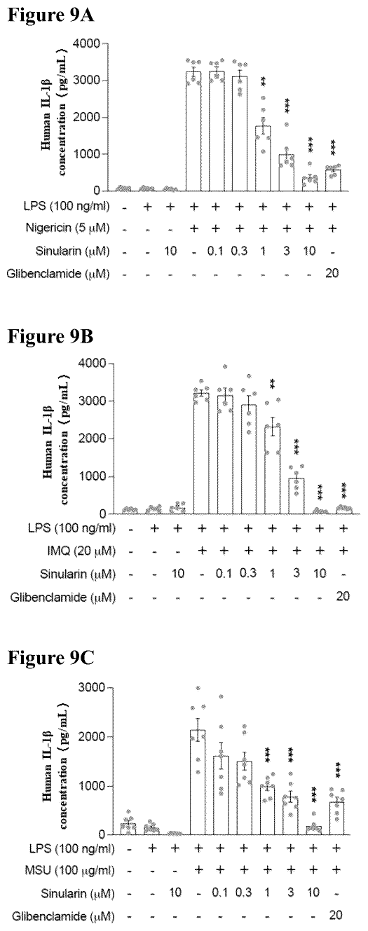

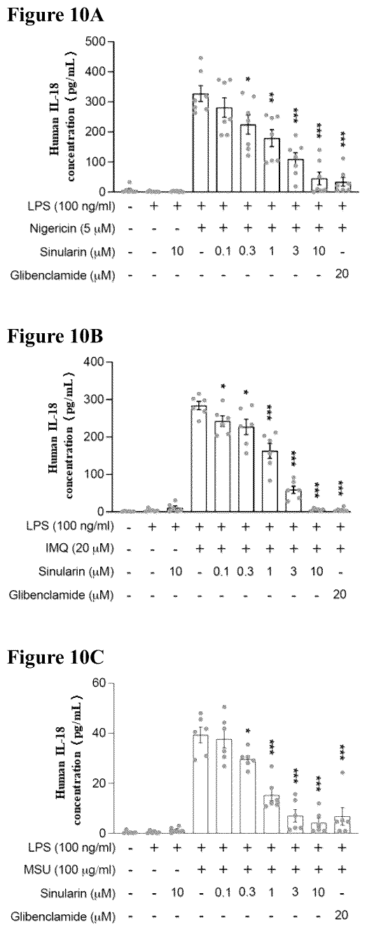

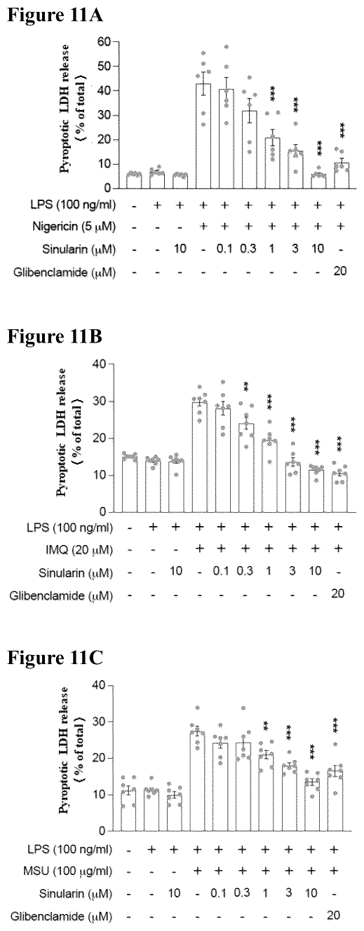

The results showed that post-treatment sinularin also significantly and dose-dependently inhibited IL-1β and IL-18 release induced by activators such as nigericin, IMQ, or MSU. The IC₅₀ are 1.26±0.27 μM (FIG. 9A), 1.62±0.23 μM (FIG. 9B), 1.19±0.32 μM (FIG. 9C), and 2.49±1.02 UM (FIG. 10A), 1.03±0.16 μM (FIG. 10B), and 0.26±0.12 μM (FIG. 10C), respectively.

### Inhibition of IL-1β or IL-18 Release Caused by NLRP3 Inflammasome by Sinularin

Sinularin (0.1 to 10 μM) was added to d-THP-1 cells after LPS priming, and the release of LDH was detected after the reaction of signal II activator.

The results showed that post-treatment sinularin also significantly and dose-dependently inhibited LDH release induced by activators such as nigericin, IMQ, or MSU. The IC₅₀ are 2.57±0.69 μM (FIG. 11A), 2.82±0.56 μM (FIG. 11B), and 1.82±0.26 μM (FIG. 11C), respectively.

### Inhibition of Morphological Changes Caused by Pyroptosis by Sinularin

Field Emission Scanning Electron Microscope (FE-SEM) was used in the present invention for the observation of cell morphological changes to confirm whether sinularin inhibited cell pyroptosis caused by NLRP3.

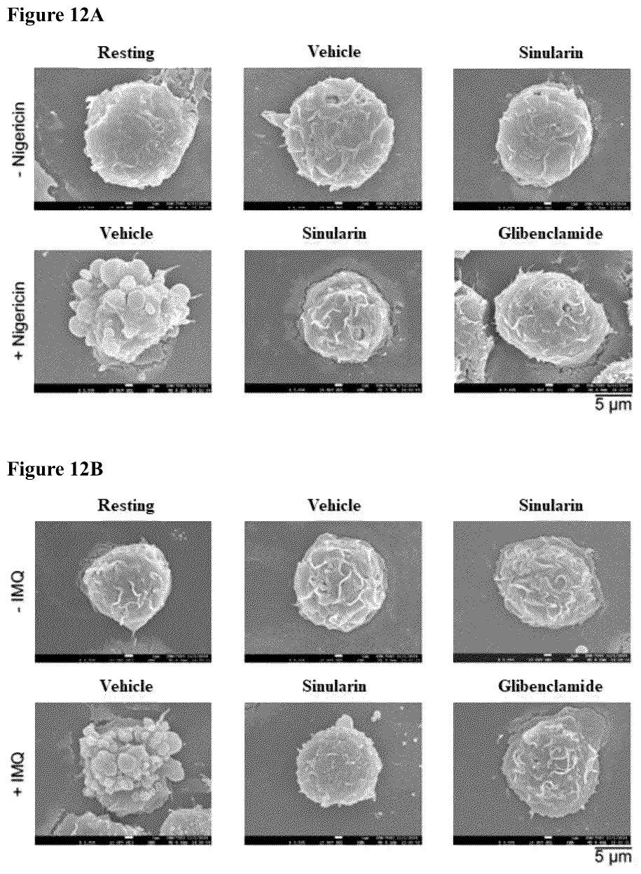

Nigericin and IMQ were used to stimulate the activation of NLRP3 inflammasomes in d-THP-1 cells, and it could be observed that the cells produced a typical pyroptosis cell morphology, including cell swelling, cell lysis, and bubble. Sinularin effectively inhibited the pyroptosis morphological changes caused by nigericin and IMQ (FIG. 12A and FIG. 12B).

### Inhibition of ASC Speck Formation by Sinularin

Upon stimulation, NLRP3 activates the signals, ASC speck occurs in activator-induced apoptosis by forming aggregates of ASC. Fluorescence calibration of ASC was used in the present invention to evaluate whether sinularin inhibits the phenomenon of ASC speck aggregation.

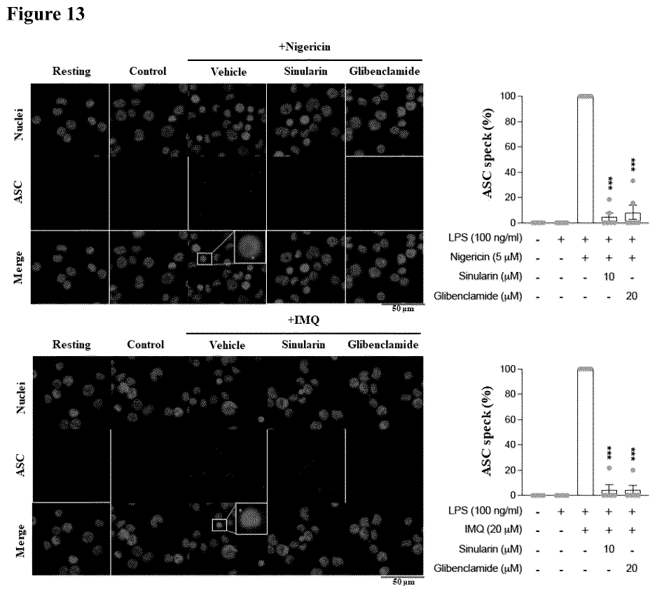

As shown in FIG. 13, ASC speck aggregation caused by activation of nigericin and IMQ was observed in vehicle group, while the ASC speck aggregation was significantly reduced by treatment with sinularin (10 μM).

### Inhibition of Reactive Oxygen Species (ROS) Generation by Sinularin

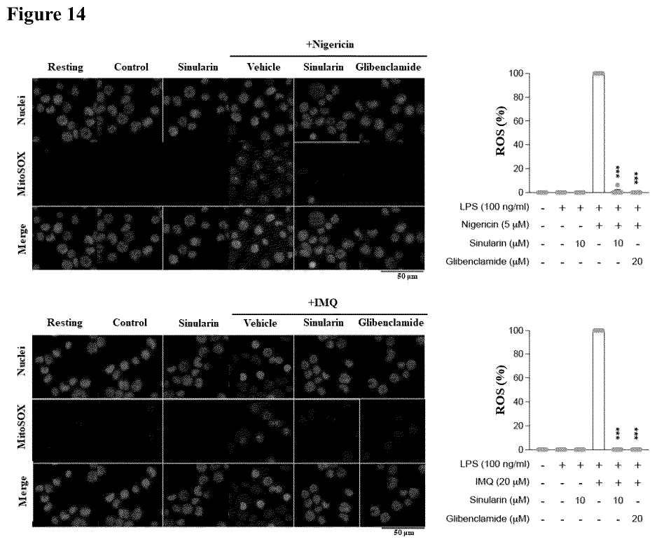

The ROS generated by mitochondria was observed by using MitoSOX fluorescence staining. As shown in FIG. 14, the result indicates that both nigericin and IMQ successfully induced ROS generation (vehicle group), and sinularin (10 μM) significantly inhibited nigericin and IMQ induced ROS production.

### Inhibition of NLRP3 Inflammasome-Related Proteins by Sinularin

Sinularin (0.1 to 10 μM) was added to d-THP-1 cells after LPS priming, and the expression of NLRP3 inflammasome-related proteins were detected after the reaction of signal II activator.

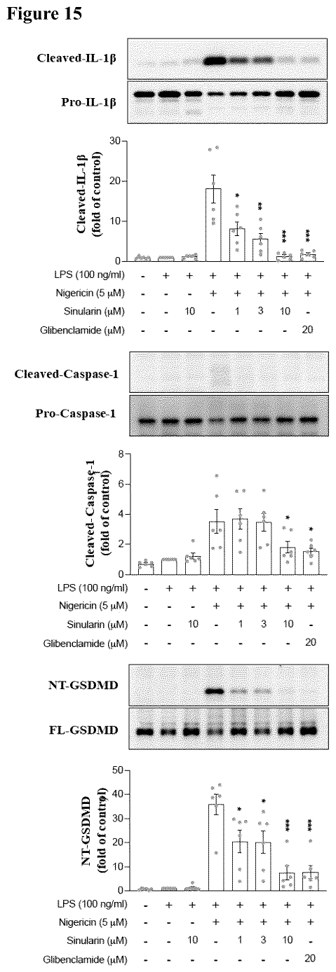

As shown in FIG. 15, the western blotting result indicates that sinularin (10 μM) significantly inhibited the increase effect of nigericin induced NLRP3 inflammasome-associated activation proteins, including cleaved-IL-1β, cleaved-Caspase-1, and N-terminal cleaved GSDMD (NT-GSDMD).

### Mitigation of Acute Liver Injury (ALI) in Mice by Sinularin

A disease model of LPS/D-GalN-induced ALI in mice was established for understanding the potential of sinularin as a lead drug.

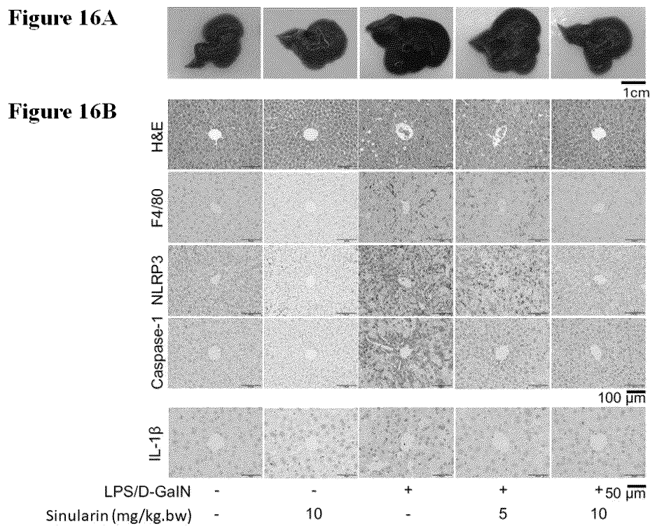

See FIG. 16A, ALI in mice were induced by intraperitoneal injection of 40 μg/kg-body weight (40 μg/kg·bw) of LPS and 500 mg/kg-body weight (500 mg/kg·bw) of D-galactosamine (D-GalN) mixture for 5 hours, significant bleeding in appearance, dark red in appearance, and enlarged were observed in the liver of ALI mice. After treatment of sinularin by tail vein injection, the liver color was light pink, indicated that the ALI phenomenon was significantly improved.

The results of hematoxylin-eosin (H&E) staining and immunohistochemistry (IHC) staining of liver tissue sections are shown in FIG. 16B.

H&E staining of liver tissue sections were analyzed, in the normal group liver sections, hepatocytes were closely arranged, and sinusoidal tubules radially surrounded the central vein; However, liver tissue was congested and had extensive infiltration of red blood cells in the liver sections of the LPS/D-GalN stimulated ALI group. In the liver sections of the sinularin treated group, it was found that liver inflammation and tissue infiltration were significantly inhibited.

IHC staining was further performed to observe the expression of macrophages and NLRP3 inflammasome-associated activated proteins in liver-damaged tissues. Significant macrophage (F4/80) infiltration and expression of NLRP3, Caspase-1, and IL-1β were observed in liver sections of the LPS/D-GalN stimulated ALI group, and a significant effect on improving ALI was observed in sinularin treated group.

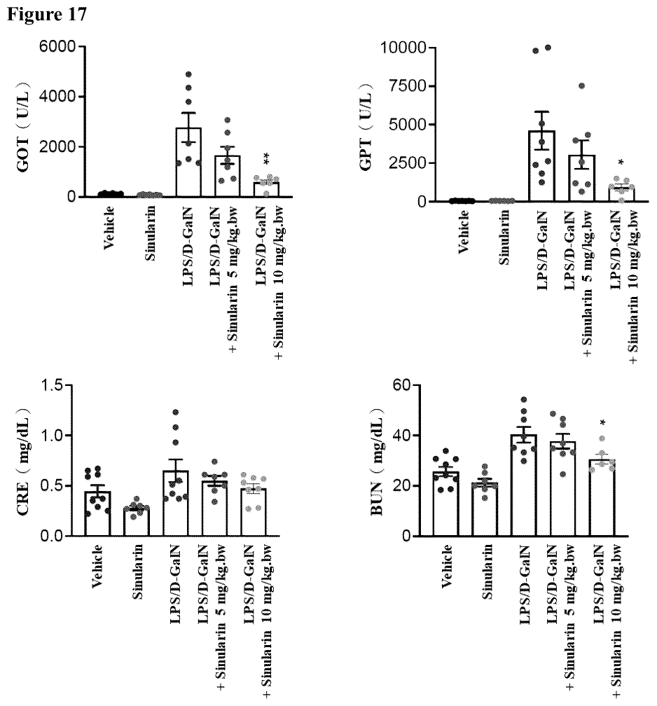

In addition, biochemical values such as blood GOT, GPT, BUN, and CRE were also measured in the present invention to be indicators for detecting damage to the liver and kidney. As shown in FIG. 17, GOT and GPT values were significantly higher in the ALI control group, while effective reductions in GOT and GPT values were observed in sinularin treated group. There was no significant difference between BUN and CRE values.

### Mitigation of Psoriasis in Mice by Sinularin

An animal disease model of IMQ cream-induced psoriasis in mice was established for understanding the potential of sinularin as a lead drug in improvement of psoriasis, and the improvement of psoriasis in each group was evaluated.

Sinularin was skin topically applied in mice in the experiment, the tested dosages were 25 mg/kg·bw (IMQ+sinularin 25) and 50 mg/kg·bw (IMQ+sinularin 50).

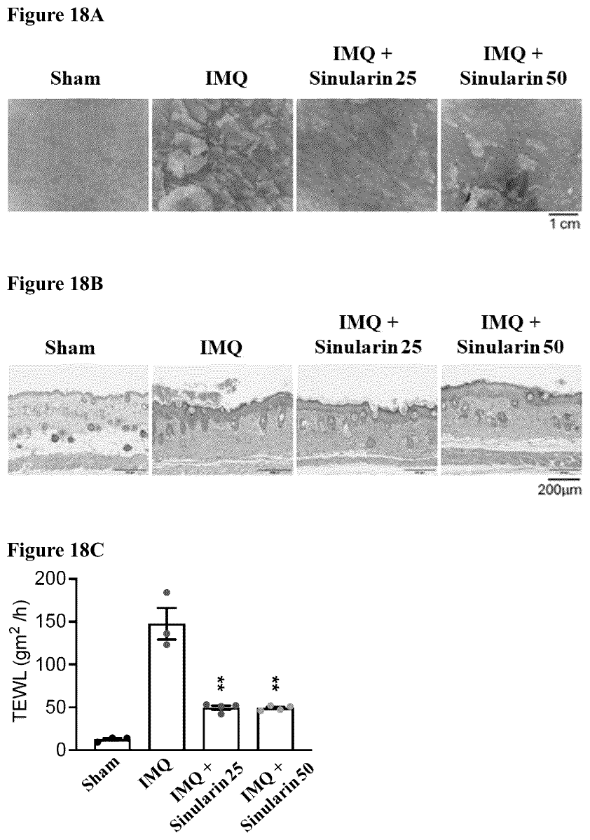

Compared with the sham group, significantly reduced the scaling of the mouse skin were observed in 25 mg/kg·bw sinularin treated group (IMQ+sinularin 25) and the 50 mg/kg·bw sinularin treated group (IMQ+sinularin 50) (FIG. 18A). An improvement in the infiltration of inflammatory cells was also shown in H&E staining of tissue sections (FIG. 18B). At the same time, it was also found that the skin of psoriasis mice trans epidermal water loss (TEWL) (FIG. 18C).

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The cells, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

What is claimed is:

1. A method for preventing or treating inflammasome-mediated inflammatory disorders, comprising administration of a cembrane-type diterpene compound to a subject in need thereof or to a subject suffering from inflammation mediated by inflammasome.

2. The method of claim 1, wherein the cembrane-type diterpene compound is cembranolide.

3. The method of claim 2, wherein the cembranolide comprises dehydrosinulariolide, dihydroaustrasulfone alcohol, dihydrosinularin, 5-epi-sinuleptolide, 11-epi-sinulariolide acetate, sinularin, sinulariolide or sinuleptolide.

4. The method of claim 3, wherein the cembranolide is sinularin.

5. The method of claim 1, wherein an effective amount of the cembrane-type diterpene compound is 0.4 mg/kg·bw to 6.1 mg/kg·bw in a 60 kg-weight human.

6. The method of claim 1, wherein an effective amount of the cembrane-type diterpene compound is 0.4 mg/kg·bw to 0.8 mg/kg·bw in a 60 kg-weight human by injection.

7. The method of claim 1, wherein an effective amount of the cembrane-type diterpene compound is 2 mg/kg to 4.1 mg/kg in a 60 kg-weight human by topical use.

8. The method of claim 1, wherein the inflammasome-mediated inflammatory disorders comprise stroke, type II diabetes (T2DM), atherosclerosis, severe acute respiratory syndrome coronavirus 2 (SARS-COV-2), acute respiratory distress syndrome (ARDS), acute lung injury (ALI), inflammatory bowel disease, atopic dermatitis, psoriasis, alopecia areata, systemic lupus erythematosus (SLE), SLE-induced lupus nephritis (LN), gouty arthritis, rheumatic disease, human autoimmune skin disease, Alzheimer's disease, Parkinson's disease, Huntington's disease, or cryopyrin-associated periodic syndrome (CAPS).

9. The method of claim 8, wherein the cryopyrin-associated periodic syndrome (CAPS) comprises familial cold autoinflammatory syndrome (FCAS), Muckle-Well syndrome (MWS), or chronic infantile neurological cutaneous articular syndrome (CINCA).

10. The method of claim 1, wherein the subject is human or non-human mammal.
